(19) United States
(12) Patent Application Publication
(10) Pub. No.: US 2025/0243144 A1
(43) Pub. Date: Jul. 31, 2025

(54) SOLID ADDITIVES FOR ORGANIC SOLAR CELLS AND METHODS OF PREPARING PHOTOACTIVE LAYERS COMPRISING THEM

(71) Applicant: GWANGJU INSTITUTE OF SCIENCE AND TECHNOLOGY, Gwangju (KR)

(72) Inventors: Soo Young JANG, Gwangju (KR); Hong kyu KANG, Gwangju (KR); Mi CHOI, Gwangju (KR); Hyeon Seok JEONG, Gwangju (KR)

(21) Appl. No.: 19/041,923

(22) Filed: Jan. 30, 2025

(30) Foreign Application Priority Data
Jan. 31, 2024 (KR) 10-2024-0015208

(51) Int. Cl. C07C 49/84; H10K 30/50; H10K 85/60

(52) U.S. Cl. CPC C07C 49/84; H10K 30/50; H10K 85/615

(57) ABSTRACT

The present inventive concept relates to a solid additive for organic solar cells and a method of preparing a photoactive layer including the same. It is possible to manufacture an organic solar cell with excellent power conversion efficiency and thermal stability by preparing a photoactive layer including a non-volatile, non-halogenated solid additive.

### CLAIM FOR PRIORITY

This application claims priority to Korean Patent Application No. 10-2024-0015208 filed on Jan. 31, 2024 in the Korean Intellectual Property Office (KIPO), the entire contents of which are hereby incorporated by reference.

### BACKGROUND

### 1. Technical Field

Example embodiments of the present inventive concept relate in general to a solid additive for organic solar cells and a method of preparing a photoactive layer including the same and more specifically to a non-volatile, non-halogenated solid additive and a method of preparing a photoactive layer of a non-fullerene acceptor-based organic solar cell including the solid additive, which can improve the power conversion efficiency and thermal stability of the organic solar cell.

### 2. Related Art

As one of the solar photovoltaic technologies among the renewable energy sources, research is being conducted on organic solar cells based on non-toxic organic materials. Organic solar cells have advantages in that low-cost mass production processes are available and translucent devices can be applied.

Organic solar cells include a photoactive layer with a bulk heterojunction structure that mixes electron acceptors and electron donors. Among them, fullerene acceptors are in the spotlight as organic solar cell materials due to their electron acceptability, electron mobility, and excellent film manufacturing properties.

However, fullerene acceptor-based organic solar cells have the disadvantage of high voltage loss during operation. Thus, research is being conducted on non-fullerene acceptor-based organic solar cells.

To manufacture high-performance bulk heterojunction organic solar cells based on non-fullerene acceptors, various additives are generally used. Among them, high-boiling-point solvent additives are mainly used to optimize the nanostructure of the photoactive layer of the cell due to their easy input and processing. Since the added molecules have selective solubility and high boiling points, excessive aggregation of small molecule receptors is delayed on a photoactive film, and thus, it is possible to create a favorable bulk heterojunction structure.

However, high-boiling-point solvent additives remain in the photoactive layer even after manufacturing a device and accelerate the instability of the cell structure, thereby lowering the stability of the device. In addition, when the solvent additive is photosensitive and forms reactive radicals, photoactive materials are rapidly decomposed, thereby reducing the photostability of the organic solar cell.

To solve the above problems of high-boiling-point additives, research on volatile solid additives through thermal annealing has been conducted. However, it is difficult to commercialize volatile solid additives because halogen elements included in volatile solid additives are toxic and harmful to the environment and the human body. They also cause morphological defects during the sublimation process, resulting in morphological instability of the device.

Therefore, in order to manufacture stable, high-performance organic solar cells based on non-fullerene acceptors, there is a need to develop the technology that can improve the performance and stability of the device without including halogen elements.

### SUMMARY

Accordingly, example embodiments of the present inventive concept are provided to substantially obviate one or more problems due to limitations and disadvantages of the related art.

Example embodiments of the present inventive concept provide a solid additive for organic solar cells.

Example embodiments of the present inventive concept also provide a method of preparing a photoactive layer including the above-described solid additive for organic solar cells.

In some example embodiments, a solid additive for organic solar cells represented by the following Chemical Formula 1 is provided,

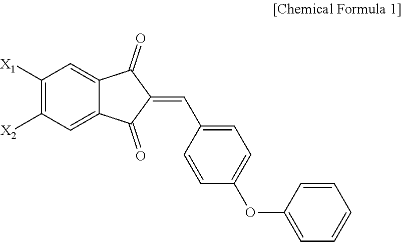

(X₁ and X₂ are each independently —H, —OCH₃, —CH₃, —CN, or —NO₂).

In other example embodiments, a method of preparing a photoactive layer including a solid additive for organic solar cells represented by the following Chemical Formula 2 is provided. The photoactive layer is formed by forming a mixed solution including an electron donor and an electron acceptor, adding a solid additive including a compound represented by the following Chemical Formula 2 to the mixed solution to form a coating solution, and coating on the electron transport layer with the coating solution.

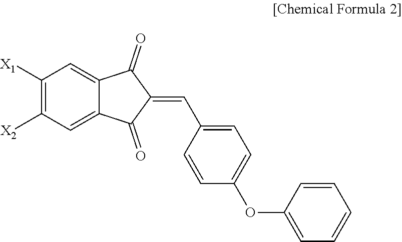

(X₁ and X₂ are each independently —H, —OCH₃, —CH₃, —CN, or —NO₂).

### BRIEF DESCRIPTION OF DRAWINGS

Example embodiments of the present inventive concept will become more apparent by describing in detail example embodiments of the present inventive concept with reference to the accompanying drawings, in which:

FIG. 1 shows a ¹H nuclear magnetic resonance (NMR) spectrum according to a preferred embodiment of the present inventive concept;

FIG. 2 shows a ¹H NMR spectrum according to a preferred embodiment of the present inventive concept;

FIG. 3 shows a graph of photovoltaic parameters according to a preferred embodiment of the present inventive concept;

FIG. 4 shows a graph of photovoltaic parameters according to a preferred embodiment of the present inventive concept;

FIGS. 5A-5D show graphs of electrochemical performance measurement results according to a preferred embodiment of the present inventive concept;

FIG. 6 shows a graph of thermogravimetric analysis (TGA) results according to a preferred embodiment of the present inventive concept;

FIGS. 7A-7B show Fourier transform infrared spectroscopy (FT-IR) spectra according to a preferred embodiment of the present inventive concept;

FIG. 8 shows a UV-Vis absorption spectrum according to a preferred embodiment of the present inventive concept;

FIGS. 9A-9C show atomic force microscopy (AFM) images according to a preferred embodiment of the present inventive concept;

FIGS. 10A-10D show images of grazing incidence wide-angle X-ray scattering (GIWAXS) measurement results according to a preferred embodiment of the present inventive concept;

FIGS. 11A-11D show graphs of time-resolved photoluminescence (TRPL) measurement results according to a preferred embodiment of the present inventive concept; and

FIGS. 12A-12C show graphs of thermal stability data according to a preferred embodiment of the present inventive concept.

### DESCRIPTION OF EXAMPLE EMBODIMENTS

Since the present inventive concept is susceptible to various modifications and alternative forms, specific embodiments thereof are shown by way of example in the drawings and will herein be described in detail. It should be understood, however, that there is no intent to limit the present inventive concept to the particular forms disclosed, but on the contrary, the present inventive concept is to cover all modifications, equivalents, and alternatives falling within the spirit and scope of the inventive concept.

Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The term “electron donor” used in the present inventive concept refers to a material that transfers electrons to other molecules or ions, and may be used interchangeably with “donor,” which has the same meaning in this specification.

The term “electron acceptor” used in the present inventive concept refers to a material that accepts electrons from other materials, and may be used interchangeably with “acceptor,” which has the same meaning in this specification.

In this specification, when a part of a layer, a membrane, a plate, etc. is said to be “on” or “above” another part, this includes not only the case where it is directly on the other part, but also the case where there is another part therebetween.

Hereinafter, various example embodiments of the present inventive concept will be described in detail with reference to the attached drawings.

### EXAMPLE

The present inventive concept provides a solid additive for organic solar cells. The solid additive may include a compound represented by the following Chemical Formula 1 and is added to the photoactive layer of an organic solar cell.

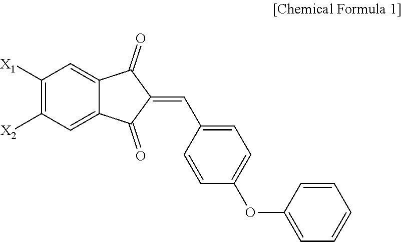

(X₁ and X₂ are each independently —H, —OCH₃, —CH₃, —CN, or —NO₂).

The solid additive may be composed of a material that does not include a halogen element. Since the solid additive is composed of non-fluorinated molecules, it is not toxic and harmful to the human body and the environment, and it is possible to prevent an increase in the morphological instability of the organic solar cell because the solid additive does not cause defects in the cell during thermal annealing of the photoactive layer. The side chains of the solid additive, X₁ and X₂, may each be independently any one of —H, —OCH₃, —CH₃, —CN, and —NO₂, but are not limited thereto, and may be any side chain used at a terminal portion of an electron acceptor.

When X₁ and X₂ in Chemical Formula 1 are both H, the solid additive is synthesized through a first step of forming 4-phenoxybenzaldehyde by adding and reacting 4-fluorobenzaldehyde, phenol, and K₂CO₃ in a solvent; and a second step of obtaining 2-(4-phenoxybenzylidene)-1H-indene-1,3 (2H)-dione (PID) by adding and reacting the 4-phenoxybenzaldehyde formed through the first step, 1H-indene-1,3 (2H)-dione (ID), and piperidine to a solvent, which may be represented by the following Scheme 1. Hereinafter, the solid additive is based on the case where X₁ and X₂ in Chemical Formula 1 are both H.

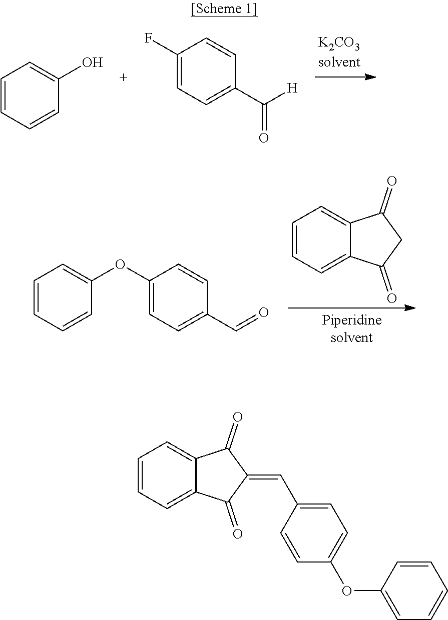 [SMILES: Oc1ccccc1 ; O=Cc1ccc(F)cc1 ; O=Cc1ccc(Oc2ccccc2)cc1 ; O=C1CC(=O)c2ccccc21 ; O=C1C(=Cc2ccc(Oc3ccccc3)cc2)C(=O)c2ccccc21]

The present inventive concept provides a method of preparing a photoactive layer included in a non-fullerene acceptor-based solar cell including the above solid additive.

The photoactive layer is prepared through a first step (S10) of forming a mixed solution including an electron donor and an electron acceptor; a second step (S20) of forming a coating solution by adding a solid additive including a compound represented by the following Chemical Formula 2 to the mixed solution; and a third step (S30) of forming a photoactive layer by coating on the electron transport layer of the organic solar cell with the coating solution formed in the second step (S20).

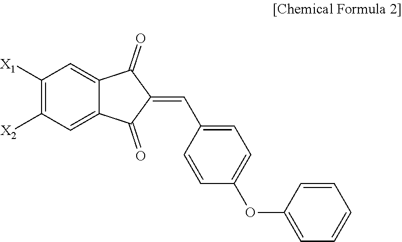

(X₁ and X₂ are each independently —H, —OCH₃, —CH₃, —CN, or —NO₂).

The first step S10 is for forming a mixed solution including an electron donor and an electron acceptor.

The electron donor may include any one selected from the group consisting of PM6, PBDB-TF, PTB7, PTB7-Th, PBDB-T, PBQx-TF, PBQx-TCI, BRT-CI, D18, and D18-Cl, and it is more preferable to use PM6 as the electron donor. However, the electron donor is not limited to the materials listed above, and any material that can transfer electrons to other molecules or ions may be used.

The electron acceptor may include any one selected from the group consisting of Y6, Y6-BO, IT-4F, ITIC, IEICO-4F, N3, L8-BO, BTP-eC9, eC9-2Cl, and PY-IT, and it is more preferable to use Y6 as the electron acceptor. However, the electron acceptor is not limited to the materials listed above, and any material that can accept electrons from the electron donor may be used.

The second step (S20) is for forming a coating solution by adding a solid additive including the compound represented by Chemical Formula 2 to the mixed solution formed in the first step (S10).

When X₁ and X₂ in Chemical Formula 2 are both H, the solid additive may be 2-(4-phenoxybenzylidene)-1H-indene-1,3 (2H)-dione (PID) and may be composed of a material that does not include a halogen element. Since the solid additive is composed of non-fluorinated molecules, it is not toxic and harmful to the human body and the environment, and it is possible to prevent an increase in the morphological instability of the organic solar cell because the solid additive does not cause defects in the cell during thermal annealing of the photoactive layer.

The solid additive may be manufactured and added through an additional step when the photoactive layer of the organic solar cell is prepared.

When X₁ and X₂ in Chemical Formula 2 are both H, the solid additive may be manufactured through a first step of forming 4-phenoxybenzaldehyde by adding and reacting 4-fluorobenzaldehyde, phenol, and K₂CO₃ in a solvent; and a second step of obtaining 2-(4-phenoxybenzylidene)-1H-indene-1,3 (2H)-dione (PID) by adding and reacting the 4-phenoxybenzaldehyde formed through the first step, 1H-indene-1,3 (2H)-dione (ID), and piperidine in a solvent, which may be represented by the following Scheme 2.

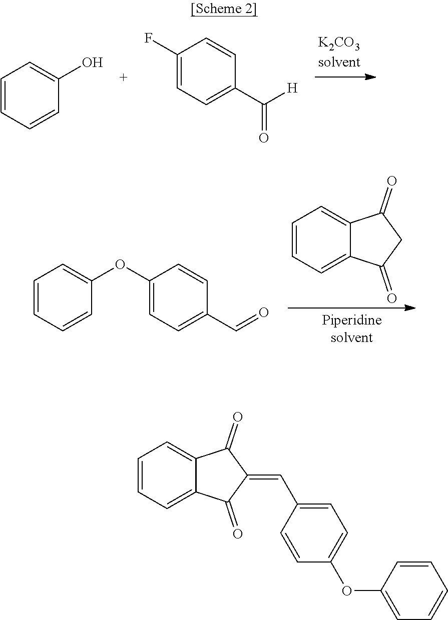 [SMILES: Oc1ccccc1 ; O=Cc1ccc(F)cc1 ; O=Cc1ccc(Oc2ccccc2)cc1 ; O=C1CC(=O)c2ccccc21 ; O=C1C(=Cc2ccc(Oc3ccccc3)cc2)C(=O)c2ccccc21]

The solid additive is preferably added in an amount of 7.0 wt % to 15.5 wt %, more preferably in an amount of 6.1 wt % to 15.3 wt %, and even more preferably in an amount of 6.3 wt % to 11.8 wt %, based on the total weight of the mixed solution. When the input amount of the solid additive is less than 7.0 wt % based on the total weight of the mixed solution, it may be difficult to achieve excellent power conversion efficiency due to the small amount of solid additive. When the input amount of the solid additive exceeds 15.3 wt % based on the total weight of the mixed solution, a large amount of additive added into the photoactive layer affects the structure and characteristics of the photoactive layer, and thus the efficiency of the cell may decrease or the electrical properties may change, reducing cell performance, and the stability of the photoactive layer may decrease due to the interaction between the photoactive layer and the additive. Therefore, it is preferable that the solid additive is added in an amount of 7.0 wt % to 15.5 wt % based on the total weight of the mixed solution.

The third step (S30) is for forming a photoactive layer by coating on the electron transport layer of the organic solar cell with the coating solution formed in the second step (S20).

After applying the coating solution on the electron transport layer to form the photoactive layer, an additional step of performing thermal annealing treatment may be included.

The thermal annealing temperature is preferably 73° C. to 97° C., and more preferably 80° C. to 90° C. When the thermal annealing temperature is below 73° C., the crystal structure of the photoactive layer may be instable, the electrical properties of the organic solar cell may be reduced as defects occur, or stability may be reduced due to a decrease in the chemical reaction rate in the photoactive layer. On the other hand, when the thermal annealing temperature is above 97° C., it may be difficult to control crystal growth due to an increase in the crystal growth rate in the photoactive layer, the electrical performance of the cell may decrease due to the change in the crystal structure, or stability may decrease due to an increase in the chemical reaction rate. Therefore, it is preferable that the thermal annealing temperature is 73° C. to 97° C.

The present inventive concept may provide a solid additive represented by the following Chemical Formula 3 and an organic solar cell including a photoactive layer prepared through a method of preparing a photoactive layer including the solid additive.

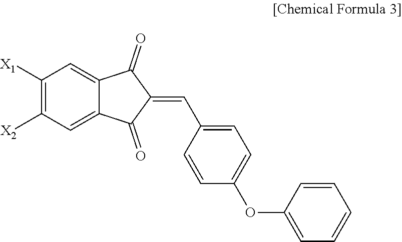

(X₁ and X₂ are each independently —H, —OCH₃, —CH₃, —CN, or —NO₂).

### Preparation Example 1: Preparation of PID

4-fluorobenzaldehyde (1.0 g, 8.00 mmol), phenol (0.83 g, 8.80 mmol), and K₂CO₃ (2.30 g, 16.00 mmol) were added to 10 mL of DMF as a solvent and stirred at 120° C. for 16 hours under N₂ atmosphere to form a mixture. After cooling the mixture to room temperature, the mixture was extracted with ethyl acetate and washed with water. Afterward, the organic layer was dried using MgSO₄ and concentrated. The concentrated organic layer was purified through silica gel column chromatography using ethyl acetate/hexane (1/10 v/v) to prepare 1.2 g of 4-phenoxybenzaldehyde in the form of a light-yellow oil. The yield was 75%.

The 4-phenoxybenzaldehyde (1.2 g, 5.47 mmol) and 1H-indene-1,3 (2H)-dione (0.80 g, 5.47 mmol) were added to 20 mL of ethanol as a solvent to form a reaction mixture. Piperidine (0.16 mL, 1.91 mmol) was added to the reaction mixture and stirred at 60° C. for 1.5 hours under N₂ atmosphere to form a mixture. After cooling the mixture to room temperature, the solvent was concentrated. Afterward, the concentrated mixture was added to ethanol, stirred for 30 minutes, and then filtered and washed to obtain a yellow precipitate. The precipitate was dissolved in 30 mL of CH₂Cl₂, filtered, and then recrystallized with CH₂Cl₂ and ethanol to obtain 1.14 g of 2-(4-phenoxybenzylidene)-1H-indene-1,3 (2H)-dione (PID) in the form of a bright yellow solid. The yield was 71.2%.

### Preparation Example 2: Manufacture of Pm6:Y6+Pid Film

PM6 (purchased from 1-Material) as an electron donor and Y6 (purchased from 1-Material) as an electron acceptor were added to chloroform at a weight ratio of 1:1.2 to form a mixed solution. The PID prepared in Preparation Example 1 was added to the mixture at 9.2 wt % of the total weight of the mixed solution, and then stirred for 2 hours to prepare a solution for preparing a photoactive layer.

A bare glass substrate was washed with deionized water, acetone, and isopropanol alcohol in a sonication bath for 15 minutes. The substrate was additionally treated with ultraviolet light/ozone (UV-O₃) for 20 minutes before solution treatment. After placing the washed substrate in a glove box filled with nitrogen, the solution for preparing a photoactive layer was applied on the surface of the substrate at 2,000 rpm for 25 seconds to form a photoactive layer, which is a PM6:Y6+PID film.

### Preparation Example 3: Manufacture of Organic Solar Cell Including PID

An ITO-coated glass substrate was washed with deionized water, acetone, and isopropanol alcohol in a sonication bath for 15 minutes. The substrate was additionally treated with ultraviolet light/ozone (UV-O₃) for 20 minutes before solution treatment. The washed ITO-coated glass substrate was oven-dried at 100° C. for more than 20 minutes. The ITO-coated glass substrate was spin-coated with ZnO and annealed in air at 200° C. for 10 minutes to form an electron transport layer. After placing the substrate on which the electron transport layer was formed in a glove box filled with nitrogen, the solution for preparing a photoactive layer of Preparation Example 2 was applied at 2,000 rpm for 25 seconds to form a photoactive layer. The photoactive layer was thermally annealed at 85° C. for 10 minutes. An organic solar cell including PID was manufactured by thermally evaporating 10 nm of MoO₃ and 100 nm of Ag on the photoactive layer and forming a positive electrode and a counter negative electrode under high vacuum.

### Comparative Example 1: Manufacture of Pm6:Y6 Film

A PM6:Y6 film was manufactured under the same conditions as in Preparation Example 2, except that PID was not added.

### Comparative Example 2: Manufacture of PM6:Y6+CN Film

A PM6:Y6+CN film was manufactured under the same conditions as in Preparation Example 2, except that 0.5 vol % of 1-chloronaphthalene (CN) was added instead of PID.

### Comparative Example 3: Manufacture of Organic Solar Cell without Additive

An organic solar cell was manufactured under the same conditions as in Preparation Example 3, except that the solution for preparing a photoactive layer of Comparative Example 1 was used instead of the solution for preparing a photoactive layer of Preparation Example 2.

### Comparative Example 4: Manufacture of Organic Solar Cell Including CN

An organic solar cell was manufactured under the same conditions as in Preparation Example 3, except that the solution for preparing a photoactive layer of Comparative Example 2 was used instead of the solution for preparing a photoactive layer of Preparation Example 2.

### Measurement Example 1

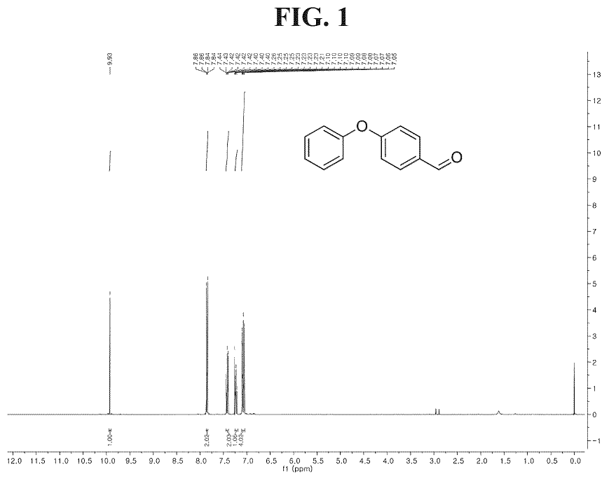

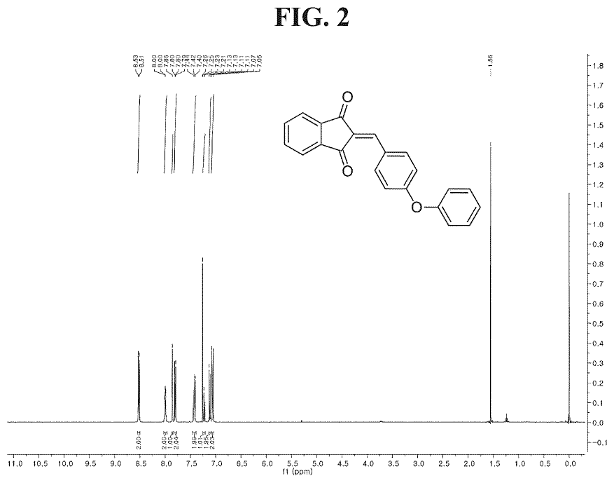

FIG. 1 shows a ¹H NMR spectrum for 4-phenoxybenzaldehyde, and FIG. 2 shows a ¹H NMR spectrum for PID. At this time, 1H NMR was measured using a 400 MHZ NMR spectrometer (JNM-ECX400 from JEOL, Ltd.).

Referring to FIG. 1, peaks were observed at 9.92 ppm, 7.86 ppm, 7.44 ppm, 7.25 ppm, and 7.10 ppm to 7.05 ppm. Therefore, it can be seen that 4-phenoxybenzaldehyde, an intermediate compound of PID of Preparation Example 1, was formed.

Referring to FIG. 2, peaks were observed at 8.55 ppm, 8.01 ppm to 7.99 ppm, 7.86 ppm, 7.82 ppm to 7.79 ppm, 7.44 ppm, 7.25 ppm, 7.13 ppm, and 7.07 ppm. Therefore, it can be seen that the PID of Preparation Example 1 was synthesized.

### Measurement Example 2

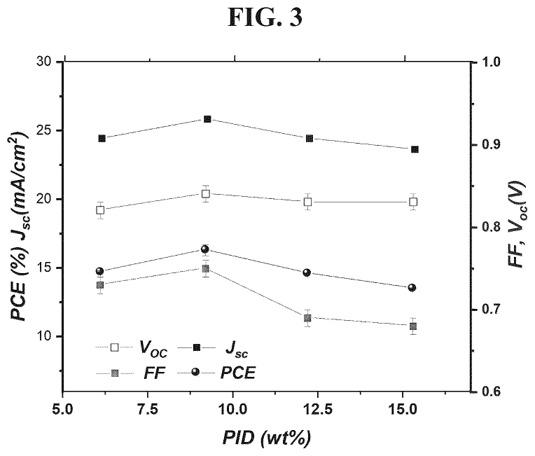

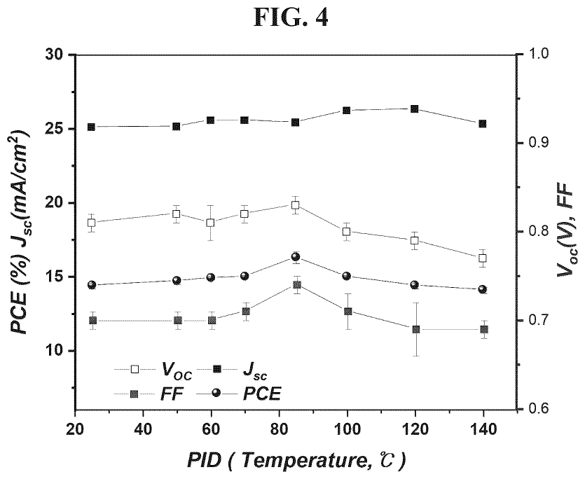

FIGS. 3 and 4 show graphs of photovoltaic parameters for the solid additive, PID, of Preparation Example 1. FIG. 3 shows a graph according to the input amount of PID, FIG. 4 shows a graph according to the thermal annealing temperature of PID, and the values are shown in Table 1 and Table 2 below.

TABLE 1

| | Additive | V_{oc} | J_{sc} | J_{cal} | | PCE |
|---|---|---|---|---|---|---|
| | content (wt %) | (V) | (mA/cm²) | (mA/cm²) | FF | (%) |
| Preparation | 6.1 | 0.81 ± 0.01 | 24.2 ± 0.2 | 24.22 ± 0.1 | 0.72 ± 0.01 | 14.6 ± 0.1 |
| Example 1 | | | | | | (14.7) |
| | 9.2 | 0.83 ± 0.01 | 25.5 ± 0.3 | 25.02 ± 0.2 | 0.74 ± 0.01 | 15.9 ± 0.4 |
| | | | | | | (16.3) |
| | 12.2 | 0.82 ± 0.01 | 24.2 ± 0.2 | 23.58 ± 0.1 | 0.68 ± 0.01 | 14.5 ± 0.1 |
| | | | | | | (14.6) |
| | 15.3 | 0.82 ± 0.01 | 23.5 ± 0.1 | 23.10 ± 0.2 | 0.67 ± 0.01 | 13.4 ± 0.1 |
| | | | | | | (13.5) |

Referring to FIG. 3 and Table 1, Table 1 shows the average values of 10 organic solar cells in which the photoactive layers formed by varying the additive content of Preparation Example 1 relative to the total weight of PM6:Y6 were thermally annealed at 85° C. for 10 minutes. When the additive content of Preparation Example 1 was 9.2 wt %, the best power conversion efficiency (PCE) was achieved with an average of 16.3%. Therefore, it is most preferable that the additive content of Preparation Example 1 is 9.2 wt %, and when the additive content of Preparation Example 1 was 6.1 wt % to 15.3 wt %, the PCE was 13.5% to 16.3% on average, indicating excellent performance.

TABLE 2

| | Annealing | | | | | |
|---|---|---|---|---|---|---|
| | temperature | V_{oc} | J_{sc} | J_{cal} | | PCE |
| | (° C.) | (V) | (mA/cm²) | (mA/cm²) | FF | (%) |
| Preparation | 25 | 0.81 ± 0.01 | 25.18 ± 0.1 | 24.63 ± 0.1 | 0.70 ± 0.01 | 14.4 ± 0.2 |
| Example 1 | | | | | | (14.6) |
| | 50 | 0.82 ± 0.01 | 25.22 ± 0.1 | 24.75 ± 0.1 | 0.70 ± 0.01 | 14.7 ± 0.2 |
| | | | | | | (14.9) |
| | 60 | 0.81 ± 0.02 | 25.65 ± 0.1 | 24.81 ± 0.2 | 0.70 ± 0.01 | 14.9 ± 0.1 |
| | | | | | | (15.07) |
| | 70 | 0.82 ± 0.01 | 25.65 ± 0.2 | 24.90 ± 0.1 | 0.71 ± 0.01 | 15.0 ± 0.1 |
| | | | | | | (15.14) |
| | 85 | 0.83 ± 0.01 | 25.5 ± 0.3 | 25.02 ± 0.2 | 0.74 ± 0.01 | 15.9 ± 0.4 |
| | | | | | | (16.3) |
| | 100 | 0.80 ± 0.01 | 26.3 ± 0.2 | 25.50 ± 0.1 | 0.71 ± 0.02 | 15.0 ± 0.1 |
| | | | | | | (15.1) |
| | 120 | 0.79 ± 0.01 | 26.4 ± 0.3 | 25.83 ± 0.1 | 0.69 ± 0.03 | 14.4 ± 0.2 |
| | | | | | | (14.6) |
| | 140 | 0.77 ± 0.01 | 25.4 ± 0.3 | 25.80 ± 0.3 | 0.69 ± 0.01 | 13.8 ± 0.2 |
| | | | | | | (14.00) |

Referring to FIG. 4 and Table 2, based on the results of FIG. 3 and Table 1, an organic solar cell was manufactured by adding 9.2 wt % of Preparation Example 1 relative to the total weight of PM6:Y6, and at this time, the thermal annealing temperature of the photoactive layer was set as shown in Table 2. The organic solar cell with the photoactive layer including 9.2 wt % of Preparation Example 1 had the best power conversion efficiency (PCE) of 16.3% on average when the thermal annealing temperature was 85° C. Therefore, it is most preferable that the thermal annealing temperature of the photoactive layer is 85° C., and when the temperature was 70° C. or above and below 100° C., the PEC was 15.1% to 16.3% on average, indicating excellent performance.

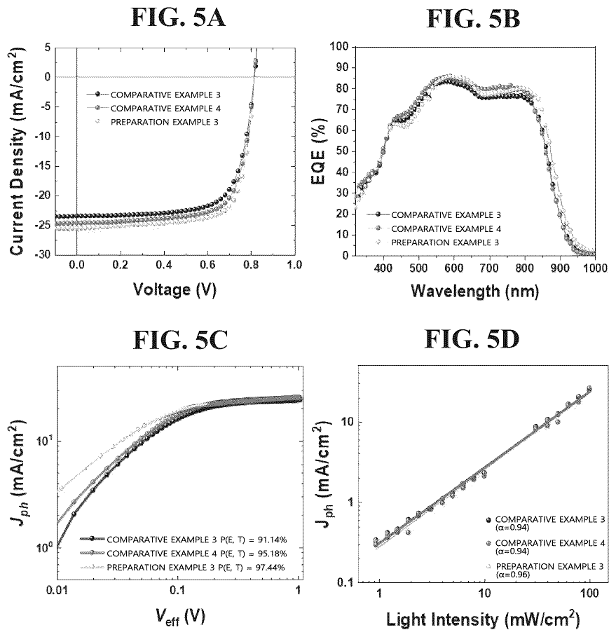

FIG. 5 shows graphs of the results of measuring the electrochemical performance of Preparation Example 3 and Comparative Examples 3 and 4, and the results are shown in Table 3 below.

TABLE 3

| | Additive | V_{oc} | J_{sc} | J_{cal} | | | PCE (%, |
|---|---|---|---|---|---|---|---|
| | content (%) | (V) | (mA/cm²) | (mA/cm²) | FF | PCE (%) | calculated) |
| Comparative | — | 0.80 ± 0.01 | 24.2 ± 0.3 | 23.79 ± 0.3 | 0.69 ± 0.01 | 13.7 ± 0.2 | 13.5 ± 0.2 |
| Example 3 | | | | | | (13.9) | (13.7) |
| Comparative | 0.5 | 0.83 ± 0.02 | 24.7 ± 0.3 | 24.70 ± 0.1 | 0.72 ± 0.01 | 15.2 ± 0.3 | 15.0 ± 0.2 |
| Example 4 | | | | | | (15.5) | (15.2) |
| Preparation | 9.2 | 0.83 ± 0.01 | 25.5 ± 0.3 | 25.02 ± 0.2 | 0.74 ± 0.01 | 15.9 ± 0.4 | 15.5 ± 0.3 |
| Example 3 | | | | | | (16.3) | (15.8) |

Referring to FIG. 5 and Table 3, the electrochemical performance in FIG. 5A was measured at an irradiance of 100 mW/cm² using a solar simulator with air mass 1.5 global (AM 1.5G).

Comparing the current density-voltage characteristics in FIG. 5A, Comparative Example 3, which did not include the additive, had a low power conversion efficiency (PCE) of 13.9% due to a current density (J_{sc}) of 24.5 mA/cm² and a low fill factor (FF) of 70%. In comparison, Comparative Example 4 in which CN was added as a conventional solvent additive had a current density (J_{sc}) of 25.0 mA/cm² and a fill factor (FF) of 73%, which were improved compared to Comparative Example 3, and thus the power conversion efficiency (PCE) slightly increased to 15.5%. In Preparation Example 3, the current density (J_{sc}) was 25.8 mA/cm², the fill factor (FF) improved to 75%, and thus the power conversion efficiency (PCE) increased to 16.3%. Therefore, it can be seen that the PID of Preparation Example 3 is superior to Comparative Example 4 using a conventional solvent additive.

FIG. 5B shows an EQE curve graph, which was measured using Oriel Quantax-300. The current density (J_{sc}) values of the EQE curve matched the current density (J_{sc}) values in FIG. 5A. In the range of 550 nm to 900 nm, Preparation Example 3 had higher EQE values than Comparative Examples 3 and 4. In the range of 830 nm to 900 nm, a red-shifted right peak and increased EQE values were observed for Preparation Example 3. This is because the PID of Preparation Example 3 enhanced charge transport and exciton dissociation due to nanoscale phase separation and improved crystallization.

FIG. 5C shows a graph illustrating exciton dissociation and charge recombination characteristics. The graph displays the photocurrent density (Jₚₕ) versus the effective voltage (V_{eff}) after measuring the saturation current density and charge dissociation probability of the cell. As a result of measuring the charge dissociation probability, the values were estimated to be 91.14% for Comparative Example 3, 95.08% for Comparative Example 4, and 97.44% for Preparation Example 3.

Therefore, Preparation Example 3 had the highest charge dissociation probability, which means that PID applied to the heterojunction layer of the photoactive layer effectively promotes dissociation of excitons into free carriers.

FIG. 5D shows a graph of evaluating the light intensity characteristics of Comparative Examples 3 and 4 and Preparation Example 3 regarding charge recombination in the cell. The estimated α value of Preparation Example 3 was 0.96, which is higher than the value of 0.94 for Comparative Examples 3 and 4. A higher a value indicates smaller bimolecular recombination losses, and thus it can be seen that the PID additive promotes exciton dissociation and suppresses charge recombination while contributing to the overall photovoltaic performance of the organic solar cell.

### Measurement Example 3

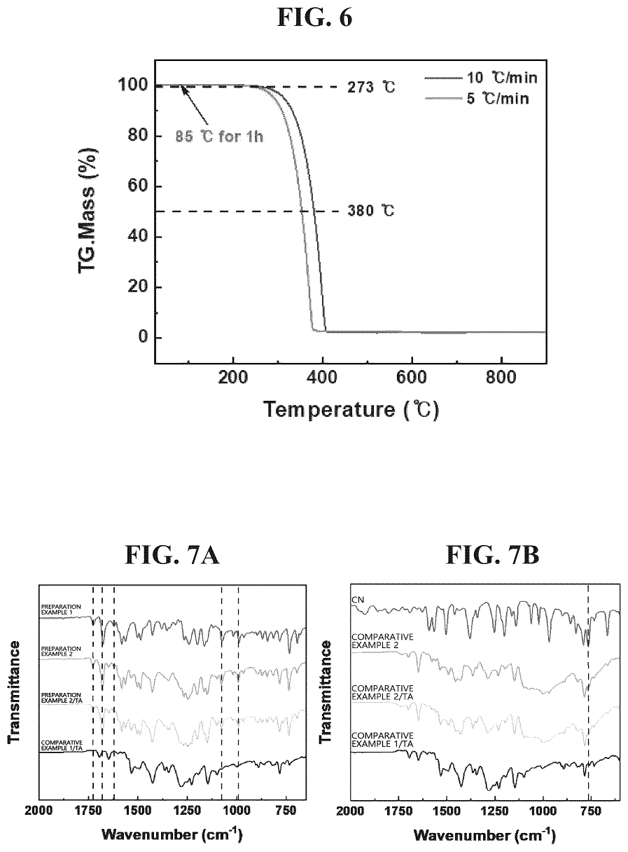

FIG. 6 is a graph showing the thermogravimetric analysis (TGA) results of Preparation Example 1.

Referring to FIG. 6, the 1% and 50% weight loss temperatures of Preparation Example 1 were 273° C. and 380° C., respectively, and when the temperature of Preparation Example 1 was maintained for 1 hour at 85° C., which is the most preferable thermal annealing temperature derived from FIG. 4, Preparation Example 1 showed no weight loss. Therefore, it can be seen that the PID of Preparation Example 1 is a non-volatile solid additive.

FIG. 7 relates to the Fourier transform infrared spectroscopy (FT-IR) spectra of Comparative Examples 1 and 2 and Preparation Example 2. Comparative Example 1/TA, Comparative Example 2/TA, and Preparation Example 2/TA mean that Comparative Examples 1 and 2 and Preparation Example 2 were each subjected to thermal annealing (TA) at 85° C. for 10 minutes.

Referring to FIG. 7, compared to Comparative Example 1, specific peaks were observed at 1725 cm-1, 1675 cm-1, 1621 cm-1, 1076 cm-1, and 991 cm-1 in Preparation Example 2, and the same peaks appeared in the thermal annealed (TA) Preparation Example 2/TA spectrum. Therefore, it can be seen that the PID of Preparation Example 2 is non-volatile and remains in the photoactive layer even after thermal annealing (TA). The peak at 765 cm-1 observed in CN, which is the additive in Comparative Example 2, was also found in the spectrum of Comparative Example 2/TA, which was thermal annealed. Therefore, it can be seen that Comparative Example 2 is also non-volatile and remains in the photoactive layer even after thermal annealing (TA).

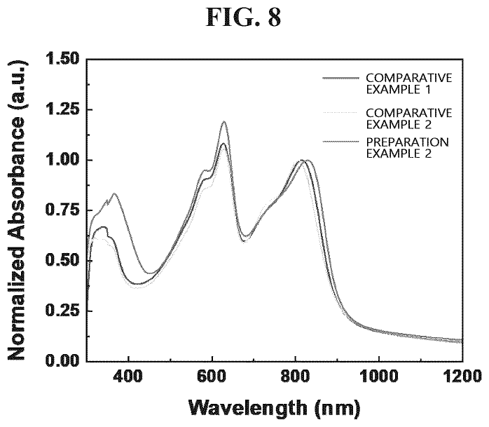

FIG. 8 shows the UV-Vis absorption spectra of Preparation Example 2 and Comparative Examples 1 and 2.

Referring to FIG. 8, compared to Comparative Example 1, shifted peaks were observed in Preparation Example 2 and Comparative Example 2 due to the additive remaining in the photoactive layer. Preparation Example 2 had an increased vibration peak intensity of the PM6 polymer at 629 nm compared to Comparative Examples 1 and 2. This was because the PID of Preparation Example 2 improved the molecular arrangement of the polymer chains. In addition, Preparation Example 2 had a 14 nm red-shifted absorption peak of the Y6 molecule at 829 nm compared to Comparative Example 1, which had a peak at 815 nm. In contrast, Comparative Example 2 had a 9 nm blue-shifted absorption peak at 806 nm compared to Comparative Example 1. Therefore, it can be seen that Preparation Example 2 which has a red-shifted absorption peak compared to Comparative Examples 1 and 2, improved the molecular arrangement of Y6.

### Measurement Example 4

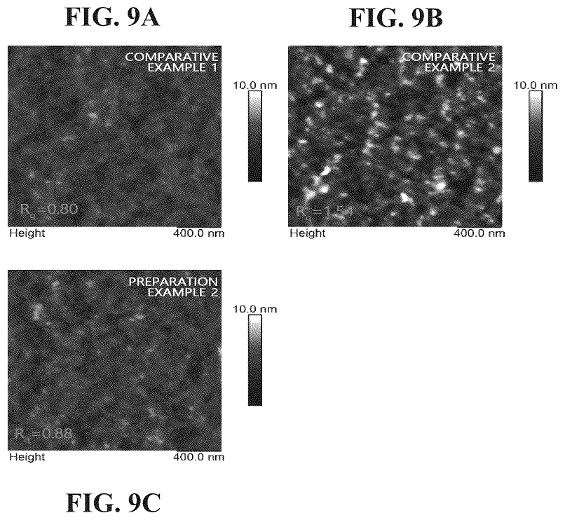

FIG. 9 shows atomic force microscopy (AFM) images of Comparative Examples 1 and 2 and Preparation Example 2. FIG. 9A is an AFM image of Comparative Example 1, FIG. 9B is an AFM image of Comparative Example 2, and FIG. 9C is an AFM image of Preparation Example 2, which were all measured after thermal annealing treatment at 85° C. for 10 minutes.

Referring to FIG. 9, Comparative Example 1 had an R_{q} value of 0.80 nm. On the other hand, Preparation Example 2 had an R_{q} value of 0.88 nm, which was slightly higher than Comparative Example 1, and distinct nanoscale phase separation was observed. Comparative Example 2 had the most uneven surface with an R_{q} value of 1.54 nm, which is presumed to be due to strong aggregation of CN and Y6 molecules.

### Measurement Example 5

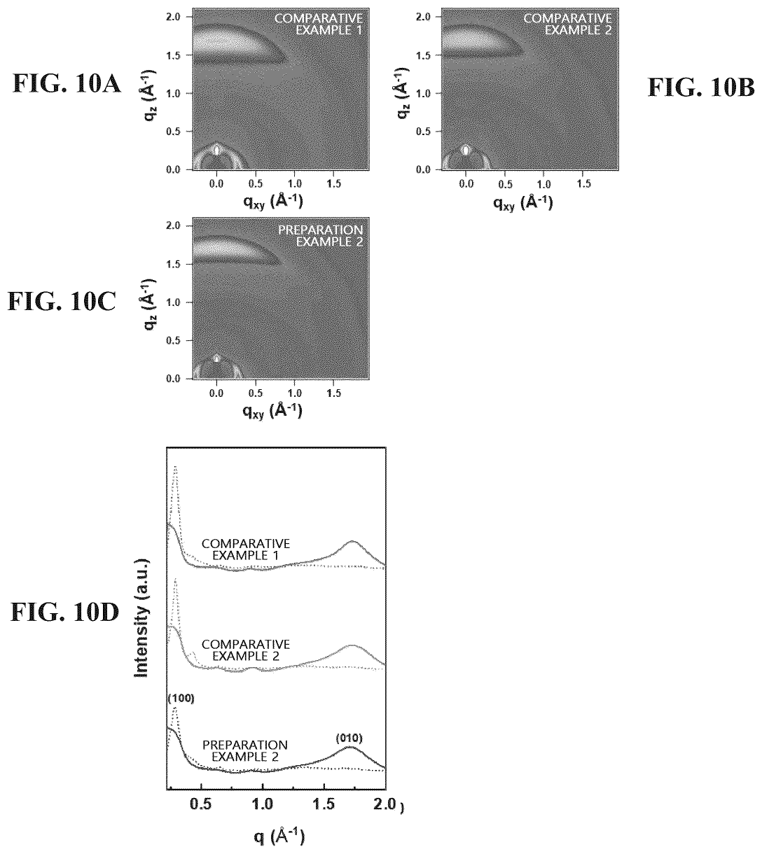

FIG. 10 shows images of the grazing incidence wide-angle X-ray scattering (GIWAXS) measurement results of Comparative Examples 1 and 2 and Preparation Example 2. FIG. 10A is a scattering profile image of Comparative Example 1, FIG. 10B is a scattering profile image of Comparative Example 2, FIG. 10C is a scattering profile image of Preparation Example 2, and FIG. 10D is a graph showing the out-of-plane and in-plane pattern lines. Before measurement, Comparative Example 1, Comparative Example 2, and Preparation Example 2 were thermally annealed at 85° C. for 10 minutes.

Referring to FIG. 10, when comparing FIGS. 10A to 10C and 10D, x-x stacking (010) diffraction peaks were observed in all films along the out-of-plane direction indicated by the solid line. For Comparative Example 1, a (100) diffraction peak was observed at q_{xy}=0.28 Å⁻¹ along the in-plane direction, and a peak was observed at q_{z}=1.70 Å⁻¹ along the out-of-plane direction. For Preparation Example 2, a (100) diffraction peak was observed at q_{xy}=0.291 Å⁻¹, and a peak was observed at q_{z}=1.73 Å⁻¹ along the out-of-plane direction, showing increased values of q_{xy} and q_{z} compared to Comparative Example 1. Through this result, it can be seen that d-spacing, which is the distance between lattice planes, decreased. This suggests that PID helps form more dense intermolecular alignment as the distance between molecules in the photoactive layer is closer.

In addition, it can be seen that the crystallinity of the photoactive layer increased through an increase in the coherence length (CL) value of the (010) peak for Preparation Example 2, calculated from the GIWAXS data using the Scherrer equation. This demonstrates that the PID of Preparation Example 2 increases the crystallinity of the photoactive material in a face-on orientation, and charge transport is facilitated through the face-on arrangement, thereby improving cell performance.

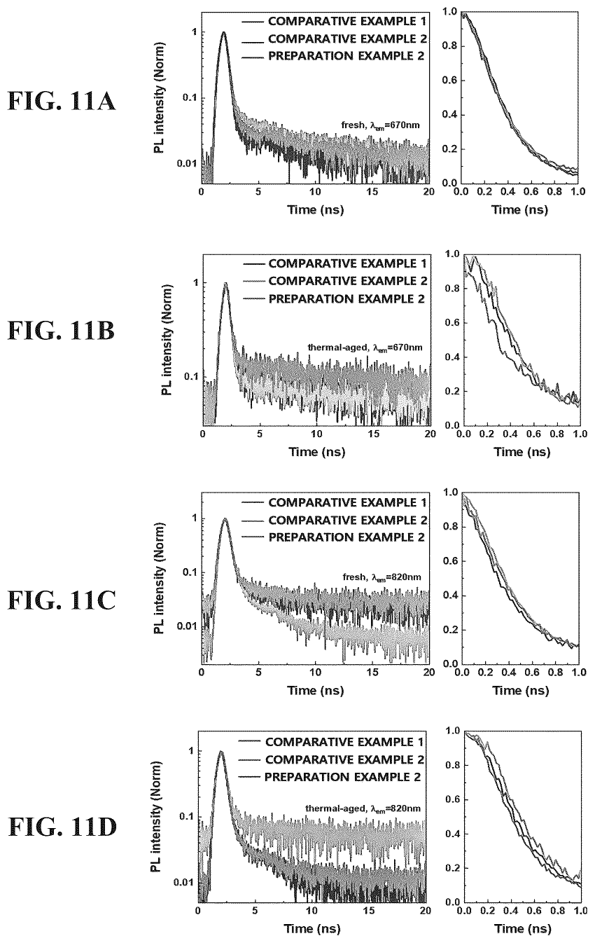

FIG. 11 shows graphs of time-resolved photoluminescence (TRPL) measurement results of Comparative Example 1, Comparative Example 2, and Preparation Example 2, and the measurement results are shown in Table 4 below. Before measurement, Comparative Example 1, Comparative Example 2, and Preparation Example 2 were thermally annealed at 85° C. for 10 minutes. Since the experiment was designed to compare the fluorescence decay lifetime of fresh films and thermally aged films, the thermally aged films were additionally heat-treated at 85° C. for 1,000 hours under an N₂ atmosphere.

TABLE 4

| | Emission | Lifetime | Film (thermally | Emission | Lifetime |
|---|---|---|---|---|---|
| Film (fresh) | (nm) | (ns) | aged) | (nm) | (ns) |
| Comparative | 670 | 0.45 | Comparative | 670 | 0.53 |
| Example 1 | | | Example 1 | | |
| Comparative | | 0.44 | Comparative | | 0.50 |
| Example 2 | | | Example 2 | | |
| Preparation | | 0.42 | Preparation | | 0.44 |
| Example 2 | | | Example 2 | | |
| Comparative | 820 | 0.43 | Comparative | 820 | 0.55 |
| Example 1 | | | Example 1 | | |
| Comparative | | 0.42 | Comparative | | 0.59 |
| Example 2 | | | Example 2 | | |
| Preparation | | 0.42 | Preparation | | 0.50 |
| Example 2 | | | Example 2 | | |

Referring to FIG. 11 and Table 4, FIGS. 11A and 11C are graphs of fresh films. FIG. 11A shows the TRPL profile at 670 nm, and Preparation Example 2 had a shorter decay lifetime compared to Comparative Examples 1 and 2. This demonstrates that efficient electron transfer occurs at the interface of PM6:Y6. FIG. 11C shows the TRPL profile at 820 nm, and here, similar decay lifetimes were observed. However, for thermally aged films in FIGS. 11B and 11D, which were measured at the same wavelength, Comparative Examples 1, 2 and Preparation Example 2 had higher ns values than fresh films. But, Preparation Example 2 showed a lower increase in the ns value compared to Comparative Examples 1 and 2. Therefore, even under external thermal stress, PID maintains a relatively stable morphology, reducing charge recombination and thus improving the thermal stability of the cell.

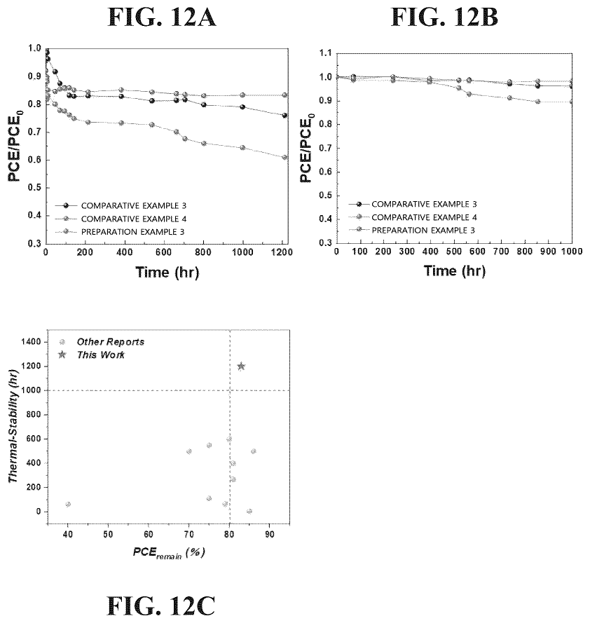

FIG. 12 shows graphs of the results of thermal stability test for Comparative Example 3, Comparative Example 4, and Preparation Example 3. For measurement, Comparative Example 3, Comparative Example 4, and Preparation Example 3 were stored on a hot plate in an N2 glove box and then subjected to thermal annealing at 85° C. (ISOS-D-2) for 1,200 hours.

Referring to FIG. 12, FIG. 12A is a graph of measuring the thermal stability of organic solar cells with a “burn-in loss” area, and FIG. 12B is a graph of measuring the thermal stability of organic solar cells without considering the “burn-in loss” area. Burn-in loss occurs due to the instability of the interface between the bulk heterojunction and MoO₃ layer. In FIG. 12B, organic solar cells in which the cause of burn-in loss was eliminated were denoted as Comparative Example 3, Comparative Example 4, and Preparation Example 3, according to the materials included in the photoactive layer, in the same manner as in FIG. 12A.

Referring back to FIG. 12, in Comparative Examples 3 and 4 of FIG. 12A, burn-in loss, in which PCE rapidly decreases within a few hours, was observed when the cell was operated. In contrast, Preparation Example 3 had excellent thermal stability with a small decrease in PCE compared to Comparative Examples 3 and 4. To confirm this more specifically, when comparing FIG. 12B which did not consider the burn-in loss area, Preparation Example 3 maintained about 98% of its initial PCE even after 1,000 hours of cell operation. Therefore, it can be seen that Preparation Example 3 including PID had excellent thermal stability.

FIG. 12C is a plot of PCE and thermal stability (hr), showing Preparation Example 3 and PM6:Y6-based organic solar cells disclosed in a previous document as control groups. The measurement values of some control groups in the red box and Preparation Example 3 are shown in Table 5 below.

TABLE 5

| | | Aging | | | |
|---|---|---|---|---|---|
| | | temperature | PCEᵣₑₘₐᵢₙ | Estimated | |
| | | (° C.) | (%) | time (hr) | Atmosphere |
| Preparation Example 3 | | 85 | 83 | 1200 | N₂ |
| Control | ITO/PEDOT/PM6/BCF/ | 65 | 75 | 550 | N₂ |
| group | Y6/PNDIT-F3N/Ag | | | | |
| | ITO/ZnO/PM6:Y6: | 100 | 80 | 600 | N₂ |
| | In₂Se₃/MoO₃/Ag | | | | |
| | ITO/ZnO/PM6:Y6 + PM6- | 80 | 86 | 500 | N₂ |
| | b-PYT6/MoO₃/Ag | | | | |

When comparing FIG. 12C and Table 5, as PM6:Y6-based organic solar cells, Preparation Example 3 and the control groups had excellent PCE values. However, the control groups had lower thermal stability than Preparation Example 3.

Therefore, it can be seen that Preparation Example 3, an organic solar cell including PID, had excellent PCE and thermal stability compared to conventional organic solar cells.

According to the present inventive concept described above, it is possible to improve the molecular arrangement of a photoactive material and control intermolecular interactions by using a solid additive for organic solar cells. Therefore, the solid additive for organic solar cells can optimize the nanoscale phase-separated morphology through the rearrangement of the photoactive material and can enhance the power conversion efficiency of the cell by improving crystallinity. In addition, since the solid additive for organic solar cells is non-volatile, it can remain in the morphology even after manufacturing the cell and optimize the morphology, and the thermal stability of the cell can be improved because the solid additive can maintain the initial power conversion efficiency at temperatures where burn-in-losses are eliminated.

Additionally, it is possible to manufacture organic solar cells with low toxicity to the human body and the environment by including a solid additive that does not use halogen elements in organic solar cells.

The solid additive including a composite of diphenyl ether (DPE) and 1H-indene-1,3 (2H)-dione (ID) can increase the conjugated polymer crystallinity of the electron donor through the DPE structure. Since the ID structure is similar to the end group of ID-rich non-fullerene acceptors, the solid additive can effectively improve x-x stacking between non-fullerene acceptor molecules and maintain a stable bulk heterojunction interface.

While the example embodiments of the present inventive concept and their advantages have been described in detail, it should be understood that various changes, substitutions and alterations may be made herein without departing from the scope of the inventive concept.

What is claimed is:

1. A solid additive for organic solar cells, which is added to a photoactive layer of an organic solar cell and represented by the following Chemical Formula 1, 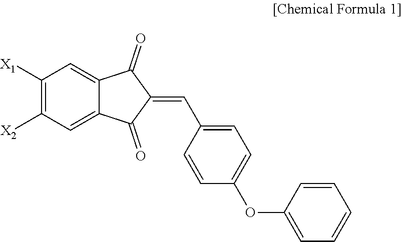
(X₁ and X₂ are each independently —H, —OCH₃, —CH₃, —CN, or —NO₂).

2. The solid additive of claim 1, wherein the solid additive is composed of non-fluorinated molecules.

3. A method of preparing a photoactive layer included in non-fullerene acceptor-based solar cells, the method comprising:
forming a mixed solution including an electron donor and an electron acceptor;
forming a coating solution by adding a solid additive including a compound represented by the following Chemical Formula 2 to the mixed solution; and
forming a photoactive layer by coating on the electron transport layer with the coating solution, 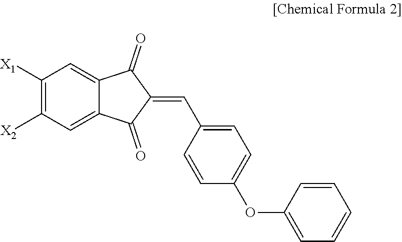
(X₁ and X₂ are each independently —H, —OCH₃, —CH₃, —CN, or —NO₂).

4. The method of claim 3, wherein the solid additive is composed of non-fluorinated molecules.

5. The method of claim 3, wherein the solid additive is added in an amount of 6.1 wt % to 15.3 wt % based on the total weight of the mixed solution.

6. The method of claim 3, wherein the photoactive layer is formed by applying the coating solution and then further performing annealing.

7. The method of claim 6, wherein the annealing temperature is 73° C. to 97° C.

8. The method of claim 3, wherein the electron donor includes any one selected from the group consisting of PM6, PBDB-TF, PTB7, PTB7-Th, PBDB-T, PBQx-TF, PBQx-TCI, BRT-CI, D18, and D18-Cl.

9. The method of claim 3, wherein the electron acceptor includes any one selected from the group consisting of Y6, Y6-BO, IT-4F, ITIC, IEICO-4F, N3, L8-BO, BTP-eC9, eC9-2Cl, and PY-IT.

10. The method of claim 3, wherein the solid additive is synthesized by forming 4-phenoxybenzaldehyde by adding and reacting 4-fluorobenzaldehyde, phenol, and K₂CO₃ in a solvent; and obtaining 2-(4-phenoxybenzylidene)-1H-indene-1,3 (2H)-dione (PID) by adding and reacting the 4-phenoxybenzaldehyde, 1H-indene-1,3 (2H)-dione (ID), and piperidine in a solvent.

11. An organic solar cell comprising a solid additive represented by the following Chemical Formula 3 in a photoactive layer, 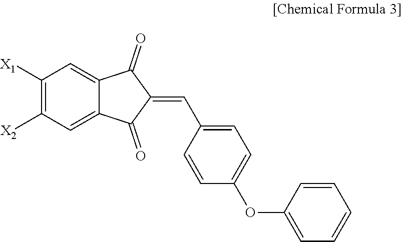
(X₁ and X₂ are each independently —H, —OCH₃, —CH₃, —CN, or —NO₂).
